(19) United States
(12) Patent Application Publication
(10) Pub. No.: US 2011/0212524 A1
(43) Pub. Date: Sep. 1, 2011

(54) BIOMATERIAL AND PREPARATION METHOD THEREOF

(75) Inventors: Horng-Ji LAI, Taipei (TW); Chien-Cheng LIN, Taipei City (TW); Shang-Ming LIN, Fongyuan City (TW)

(73) Assignee: BODY ORGAN BIOMEDICAL CORPORATION, Taipei City (TW)

(21) Appl. No.: 13/019,134

(22) Filed: Feb. 1, 2011

Related U.S. Application Data
(63) Continuation-in-part of application No. 12/081,015, filed on Apr. 9, 2008, now abandoned.
Application No. 12/081,015 is a continuation-in-part of application No. 11/882,328, filed on Jul. 31, 2007, now Pat. No. 7,838,038.
(60) Provisional application No. 60/868,409, filed on Dec. 4, 2006.

(51) Int. Cl. C12N 5/07; B08B 3/00

(52) U.S. Cl. 435/395; 134/26

(57) ABSTRACT

A method for preparing a biomaterial comprising the steps of acellularizing the fish scale to remove cell components, decalcifying the fish scale; and cleaning the fish scale, wherein in the step of acellularizing, it further including stirring constantly the fish scale in a solution comprised of an octylphenoxypolyethoxyethanol, a tris-buffered salt and a protease inhibition; and rinsing in a Hanks' buffered saline and the fish scale keeps the naturally 3-dimension microstructure after the step of acellularizing the fish scale.

This patent invention is a Continuation-in-part (CIP) of U.S. invention Ser. No. 12/081,015 filed Apr. 9, 2008.

### FIELD OF THE INVENTION

The present invention relates to a method for preparing a biomaterial and its invention, particularly a method for preparing a biomaterial prepared from fish scales as the scaffold for use in corneal regeneration.

### BACKGROUND OF THE INVENTION

Millions of people worldwide are blind from corneal disease or damage. Accordingly, scientists have attempted to use animal corneas to treat corneal diseases in humans and surgical treatment for corneal transplantation is carried out.

However, transplantation of a cornea not only has difficulties such as the shortage of donor corneas, but immunological rejection by aggressive immune responses often leads to failures in the transplantation. Therefore, it must to find other way to solve the problems of donor corneas.

The development of artificial corneas (keratoprostheses) is a promising alternative to obtain tissue replacements for corneal transplantation. Especially to patients who are blind due to corneal defects, artificial corneas could potentially benefit and the demand is increasing.

There are many kinds of material used as artificial corneas material nowadays. For example, the artificial corneas material made of collagen fiber, hydroxyapatite (HAP) or tri-calcium phosphate (TCP) are with great biocompatibility and safety. However, these biomaterials have disadvantages such as low mechanical strength, risk of chemical residue in cross linking, terrestrial animal transmitted disease. Therefore, these biomaterials are not suitable for scaffolds used as the artificial cornea in tissue engineering.

Besides, the artificial cornea material made of monomers is also not suitable since it fails to provide satisfactory yield and purity while retaining advantageous transparency and oxygen permeability. Thus, there still remains a need for an effective artificial cornea that can be harvested from animal corneas.

Therefore, it is desirable to develop a biomaterial having a high mechanical strength, low possibility of contracting with the terrestrial contagious disease and is desirable to develop a biomaterial applicable for the design of corneal prostheses.

### BRIEF SUMMARY OF THE INVENTION

It is an aspect of the invention to provide a method for preparing a biomaterial comprising the steps of acellularizing the fish scale to remove cell components, decalcifying the fish scale; and cleaning the fish scale, wherein in the step of acellularizing, it further including stirring constantly the fish scale in a solution comprised of an octylphenoxypolyethoxyethanol, a tris-buffered salt and a protease inhibition; and rinsing in a Hanks' buffered saline and the fish scale keeps the naturally 3-dimension microstructure after the step of acellularizing the fish scale.

It is an aspect of the invention to provide a method for preparing a biomaterial comprising the steps of acellularizing the fish scale, decalcifying the fish scale, and cleaning the fish scale and then extruding the fish scale.

The process further comprises a step of dehydrating the fish scale until the fish scale contains less than about 50% of water and a step of soaking the scale. In an embodiment of the invention, the fish scale contains less than about 25% of water.

It is another aspect of the invention to provide a method for preparing a biomaterial which comprises subjecting the fish scale to a heat treatment at a temperature of less than about 200° C.

It is yet another aspect of the invention to provide a use of the biomaterial prepared by the process described above for repairing tissues.

It is yet a further aspect of the invention to provide a method for preparing a biomaterial prepared from fish scales as the scaffold for use in corneal regeneration.

It is yet a further aspect of the invention to provide a method for corneal regeneration, comprising steps of acellularizing the fish scale, wherein the fish scale keeps the naturally 3-dimension microstructure after the step of acellularizing the fish scale; cleaning the fish scale; decalcifying the fish scale to serve as a scaffold; harvesting a corneal; seeding the corneal onto the scaffold in a plate; and culturing a cell of the corneal, wherein in the step of acellularizing, it further includes stirring constantly the fish scale in a solution comprised of an octylphenoxypolyethoxyethanol, a tris-buffered salt and a protease inhibition; and rinsing in a Hanks' buffered saline.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a flow chart illustrating process for preparing a biomaterial with simple contents.

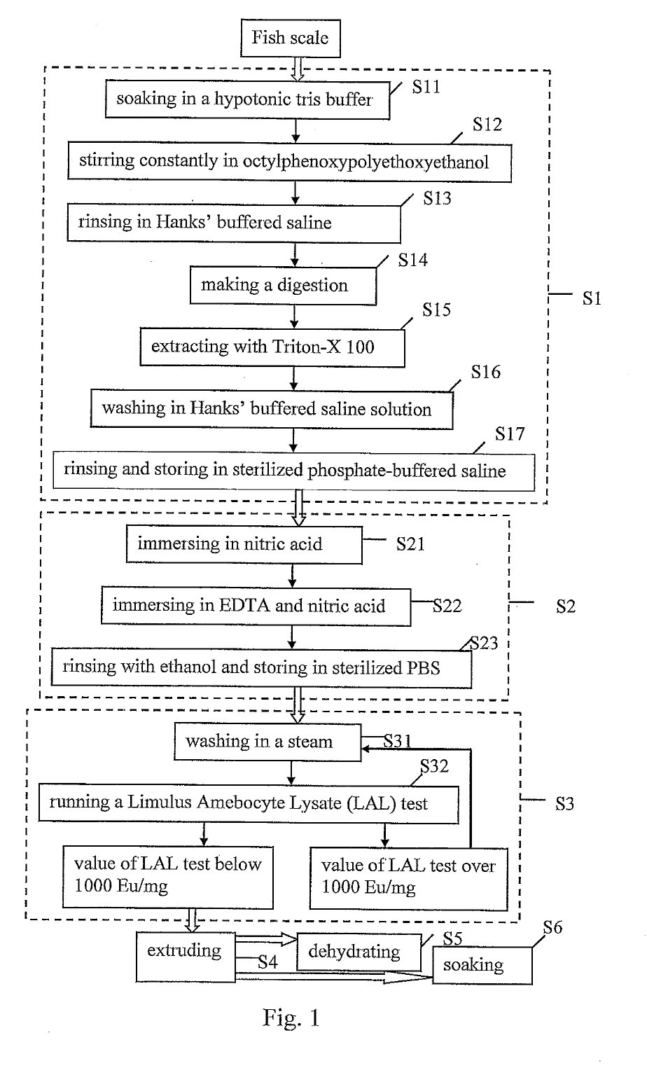

FIG. 2 is a flow chart illustrating process for preparing corneal regeneration with simple contents.

FIGS. 3A-3C are SEM micrographs of the acellularized fish scale.

FIGS. 4A-4D are micrographs of the corneal cells cultivated on the scaffold after different time periods of cultivation.

FIGS. 5A-5D are micrographs of the corneal cells cultivated on the acellular scaffold.

FIG. 6 is a picture showing the quantification of corneal cell growth on the scaffold.

### DETAILED DESCRIPTION OF THE INVENTION

Reference will now be made in detail to the present embodiments of the invention, examples of which are illustrated in the accompanying drawings.

The process for preparing a biomaterial from the fish scale in the present invention comprises acellularizing the fish scale (S1), decalcifying the fish scale (S2), and cleaning the fish scale (S3).

In accordance with some examples of the invention, before acellularizing the fish scale (S1), the fish scale may be freshly provided in a chilled or frozen manner. In a specific example of the invention, the fish scale has an average size less than about 20 cm in diameter may be selected for preparing the biomaterial. In detail, the fish scales are from a bony fish (Osteichthyes).

The step of acellularizing the fish scale (S1) is to remove cell components from cellular materials so as to avoid residual cells affecting the biocompatibility since residual cellular components and lipids within processed tissue may promote undesired effects, such as calcification and immune response. In one embodiment of the invention, the step of acellularizing the fish scale (S1) which further includes following steps. Step (S11) is to soak the fish scale in a hypotonic tris buffer (about pH 8.0) that contains a protease inhibitor (phenylmethyl-sulfonyl fluoride, 0.1˜10 mM, preferably 0.35 mM) for 24 hours at 4° C. with constant stirring. Then, Step (S12) is to stirring constant the fish scale in a surfactant, wherein the solution is preferably comprised of an about 0.1˜5% solution of octylphenoxypolyethoxyethanol (Triton X-100) in tris-buffered salt solution with protease inhibition for 24 hours at 4° C. After the step of (S12), rinsing the fish scale (S13) is performed thoroughly in a surfactant, preferably in Hanks' buffered saline solution (HBSS). After the step of (S13), making the fish scale digestion (S14) with DNase and RNase is performed at 37° C. for 1 hour in order to increase pore sizes and porosity within the test samples. This is followed by a step of extracting the fish scale (S15) with Triton-X 100 in tris buffer for 24 hours. Then, washing the fish scale (S16) is performed after the step of extracting the fish scale (S15). In the step of washing the fish scale (S16), the fish scale is washing for 48 hours in Hanks' buffered saline solution. It could extract the fish scale using an about 0.1˜10%, preferably 1% sodium dodecyl sulfate (SDS). Then, rinsing and storing the fish scale (S17) are performed in sterilized phosphate-buffered saline (PBS, pH 7.4). The step of S14 is optional, that is, if it is really clean enough, the step of S14 could not be performed.

After the step of acellularizing the fish scale (S1), since the fish scale are from a bony fish (Osteichthyes), the step of decalcifying the fish scale (S2) would be performed. In one embodiment of the invention, the step of decalcifying the fish scale (S2) which further includes following steps. Step (S21) is to immerse the fish scale in 1˜20%, preferably 5% nitric acid for 6˜16 hours at room temperature (RT). Step (S22) is to immerse the fish scale in an acid solution, preferably in 300 ml of Solution A (5˜20% Ethylenediaminetetraacetic acid (EDTA), 0.5˜5%, preferably 2% nitric acid) for 2-3 days at 4° C. with renewal of Solution A daily depending on the degree of mineralization of the fish scale. Step (S22) is to rinse the fish scale with 70˜75% ethanol and stored (S23) in sterilized PBS at 4° C. Wherein, the EDTA in an amount of 5-20 weight/volume %, preferably 10%, and at pH of 5.0 to 8.5, preferably at about 7 to 7.4.

In the step of decalcifying the fish scale (S2), it could also be performed by using the solution such as 5%-10% nitric acid in distilled water or 5-20%, preferably 10% HCl in distilled water or Plank-Rychlo's solution or Morse's solution or 0.1˜10%, preferably 5% formic acid (FA) in distilled water or 5˜20%, preferably 10% EDTA (pH 5.5˜8.5, preferably 7.4), or 5˜20%, preferably 10% EDTA/TRIS-HCl (pH 5.5˜8.5, preferably 7.4), or 5˜20%, preferably 10% EDTA with 0.01˜0.2%, preferably 0.07% (w/v) glycerol (pH 5.5˜8.5, preferably 7.4). Wherein, the Plank-Rychlo's solution is comprised of 0.1˜0.5 M, preferably 0.3 M aluminium chloride, 0.5˜10%, preferably 3% HCl, and 1˜10%, preferably 5% formic acid; and the Morse's solution is comprised of 5˜20%, preferably 10% sodium citrate, 5˜40%, preferably 20% formic acid.

The benefits of decalcifying the fish scale (S2) are such as it can increase transparency and preserving the amazing nature structure of fish scale with more special properties such as physical properties, for example mechanical strength, chemical properties and special microstructure, not to extract collagen from fish instead.

After the step of decalcifying the fish scale (S2), the step of cleaning the fish scale (S3) could be performed so as to get rid of the impurities of the fish scale. In one embodiment of the invention, the step of cleaning the fish scale (S3) which further includes following steps. Step (S31) is to wash the fish scale in a steam (S31) with other cleaning agents including but not limited to surfactant, detergent, warm water and polar solvent such as ethanol at about 60° C. Step (S32) is to run the Limulus Amebocyte Lysate (LAL) test which is an assay for detection and quantitation of bacterial endotoxin. Then, repeating the step of washing the fish scale (S31) and running the LAL test (S32) are performed until a value of LAL of the fish scale is below 200 Eu/g. Even more preferred fish scale of the LAL test is less than 50 EU/g, and most preferably less than 20 EU/g. As long as the fish scale could be cleaned enough to pass the LAL test, the present invention is not limited to any particular cleaning step.

In step of running the LAL test (S32), various analyzed technique could be used for determining the LAL value of the fish scale such as gel-clot, chromogenic technique, endpoint-turbidimetric technique or kinetic-turbidimetric technique. In one embodiment of the invention, gel-clot technique could be chosen and the protocol is shown as following.

Each assay should include both a positive control and a negative control. LAL Reagent Water can be used as a negative control. The step of running the LAL test (S32) which further includes following.

Step 321: Carefully dispense 0.1 ml of LAL solution into the Endotoxin-free vials. Label them as negative control, positive control, and samples.

Step 322: Carefully transfer 0.1 ml of positive control, negative control and the test samples to the LAL reagent in step (1). Cap the vials and mix them thoroughly.

Step 323: Place all the vials in the incubation rack and incubate the vials by placing the rack in a 37° C. non-circulating hot water or Oven.

Step 324: Remove the rack after 60 minutes (±2 minutes) of incubation, invert each vial and check whether a gel is formed or not.

a) A positive reaction is characterized by the formation of a firm gel that remains intact when the vial is inverted.

b) A negative reaction is characterized by the absence of a solid clot. The lysate may show an increased turbidity or viscosity. This is considered a negative result.

It is noted that the method for preparing the biomaterial from the fish scale of the present invention, wherein the steps of acellularizing the fish scale (S1), decalcifying the fish scale (S2), and cleaning the fish scale (S3) can exchange the order mutually. It will have six kinds of embodiment, and all of them can achieve the purpose of the present invention. No matter what kind of the order of the three steps (S1), (S2), (S3) proceed, the step of extruding the scale (S4) should proceed after the three steps (S1), (S2), (S3) totally are complete.

It could further include a step of crosslinking the biomaterials. It can be achieved physically by heating or chemically by adding with a cross linker at an optimal concentration before extruding the fish scale (S4). The cross linker is reactive with the amines group or other reactive group in the biomaterials.

In the step of extruding the scale (S4), the fish scale could be cold pressed with a pressure of more than 100 g in 2.5 cm², preferably, more than 1 kg in 2.5 cm², are submitted to hot pressing performed at a temperature of less than about 200° C. in a desired mold. One skilled in the art may also adopt other heat treatments such as thermal extrusion of any type, thermal pressing and molding steps to produce the biomaterial. The heat treatment in the present invention is not limited to the step of extruding the fish scale (S4) described above. The fish scale keeps the naturally 3-dimension microstructure after the step of extruding the fish scale (S4).

After the step of extruding the fish scale (S4), the step of dehydrating the fish scale (S5) or the step of soaking the fish scale (S6) would be performed. In the step of dehydrating the scale (S5), the fish scale would be dehydrated by air spraying, oven, freeze drying, soaking in the ethanol or other polar organic solvent or any other conventional dehydration methods available so far. The step of dehydrating the fish scale (S5) may be subjected to the step of extruding the fish scale (S4) with or without one or more cross linking ingredients. In one other example, the step of dehydrating the fish scale (S5) could be subjected to a heat treatment, such as a extruding the fish scale (S4), process performed at a temperature of less than about 200° C., preferably, about from 110° C. to 200° C. The fish scale are dehydrated until their water content is less than about 50%, preferably less than about 25%. In the step of soaking the fish scale (S6), the fish scale would be soaked in isotonic solution of target tissue, such as 0.1˜2% normal saline.

The fish scale could be dehydrated (S5) and soaked the fish scale (S6) (but not limited) after the three steps (S1), (S2), (S3) totally are complete, too. These products may be further processed, for example, by steps such as (S4), (S5), (S6), fully or partially drying and sterilizing to yield sterilized the fish scale of the biomaterials. In a preferred embodiment, these steps may be performed with or without heating. Wherein, the steps of (S4), (S5), (S6) are optional steps.

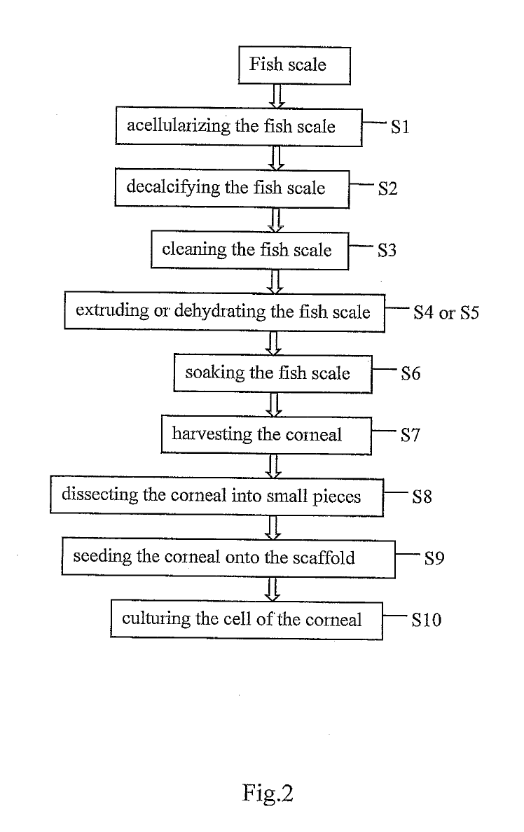

Referring to FIG. 2, it shows the method for corneal regeneration. The method comprises steps of acellularizing the fish scale (S1), decalcifying the fish scale (S2) to serve as the scaffold, cleaning the fish scale (S3), extruding the fish scale (S4), dehydrating the fish scale (S5), harvesting a corneal (S7), dissecting the corneal into small pieces (S8), seeding the corneal onto the scaffold in a plate (S9); and culturing a cell of the corneal (S10). Wherein, the steps of (S4), (S5) are optional steps in the method for corneal regeneration.

As mentioned above, in the step of acellularizing the fish scale (S1), it further includes steps such as (S11), (S12), (S13), (S14), (S15), (S16) and (S17). Besides, in the step of decalcifying the fish scale (S2), it further includes steps such as (S21), (S22) and (S23); and the step of cleaning the fish scale (S3) would also include the steps such as (S31) and (S32). In a preferred embodiment, the steps are performed as (S11), (S12), (S13), (S14), (S15), (S16) and (S17) in order, the step of decalcifying the fish scale (S2) is performed to reduce the possibility of calcification which was most probably occurred during initial cell migration at the time of in vivo use, and washing the fish scale (S31) and running the LAL test (S32) are performed repeating until a value of LAL of the fish scale is below 200 Eu/g.

After the three steps (S1), (S2), (S3) totally are complete, the fish scale serves as the scaffold and harvesting the corneal (S7) could be performed. In the step of harvesting the corneal (S7), the corneal from an animal would be rinsed with saline containing 200˜400 U/ml penicillin and 0.3 mg/ml streptomycin and preserved at 4° C. in Dulbecco's modified Eagle's medium (DMEM) supplemented with 50˜200 U/ml, preferably 100 U/ml penicillin, 0.01˜0.5 mg/ml, preferably 0.1 mg/ml streptomycin and 0.1˜0.5 g/ml, preferably 0.25 g/ml amphotericin B until use, wherein the corneal could be harvested from autologous transplantation, allogeneic transplantation or heteroplastic transplantation. Then, the step of dissecting the corneal into small pieces (S8) is performed. After the step of dissecting the corneal into small pieces (S8), seeding the corneal onto the scaffold in a plate (S9) is performed wherein the corneal would be placed one piece per well in 48-well plates.

Finally, culturing the cells of the corneal (S10) is performed by growing in DMEM supplemented with about 5˜20%, preferably 10% fetal calf serum, 1˜10 mM, preferably 4 mM L-glutamine, 5˜40 mg/ml, preferably 24 mg/ml adenine and 1% antibiotic solution. In the step of culturing the cells of the corneal (S10), the cells would be incubated at 37° C. in a humidified atmosphere with about 5% CO² in air and the culture medium would be refreshed every 2 days. In one embodiment of the invention, culturing the cells of the corneal (S10) would be performed at 37 □ in 5% CO² and water-saturated atmosphere in Eagle's minimum essential medium supplemented with 10% heat-inactivated fetal bovine serum, 0.5˜2 mg/ml, preferably 1.5 mg/ml sodium bicarbonate, 0.05˜0.2 mg/ml, preferably 0.11 mg/ml sodium pyruvate, 50˜200 U/ml, preferably 100 U/ml penicillin, 0.01˜0.5 mg/ml, preferably 0.1 mg/ml streptomycin and 0.1˜0.5 g/ml, preferably 0.25 g/ml amphotericin B. For nuclei staining and counting, plating the cells onto the scaffolds would be performed, wherein it would be placed one piece per well in 24-well plates at the density of 0.1˜10 cells/well, preferably 1×10⁵ cells/well and would be cultured for the indicated time periods. After the step of culturing the cells of the corneal (S10), the scaffold samples would be rinsed in PBS, fixed in 1˜10%, preferably 4% paraformaldehyde in PBS, permeabilized with 0.01˜0.5%, preferably 0.1% Triton X-100 and incubated with 4′, 6 diamidine-2′ phenylindole dihydrochloride (DAPI) for 20 min to label nuclei.

### Scanning Electron Microscopy

Scanning electron microscopy was used to examine the morphological characteristics of corneal cells cultured onto the acellular decalcified scaffold. Corneal debris were plated on the scaffolds and cultured for 1, 2, 3, and 7 days. Loosely adherent and unbound cells were removed from the culture wells by aspiration and the wells were washed twice with PBS. The remaining attached cells were fixed in 0.5˜5%, preferably 2.5% glutaraldehyde in PBS (pH 7.4) for 10 min. The fixative was then aspirated. After being washed in PBS, scaffolds were dehydrated in a graded series of ethanol solutions. After critical point drying, the samples were sputtered with gold using a SEM coating system, and the probes were examined by scanning electron microscopy.

### Confocal Microscopy

Cells grown on the scaffolds for the indicated time periods were washed with PBS and fixed in 3.7% formaldehyde for 15 minutes at room temperature and then permeabilized with 1% Triton X-100 for 5 minutes. After washing, cells were blocked with 10% normal goat serum and 5% bovine serum albumin in PBS for 1 hour at room temperature and incubated with Hoechst 33342 in staining buffer (1% NGS and 1% BSA in PBS) for 20 minutes at room temperature to visualize the nucleus. F-actin was visualized using Alexa Fluor 488 phalloidin (1:300). Image quantification of scaffold area and cell area was accomplished by confocal microscopy. Follows are the results of the cells culturing onto the scaffold.

### Characterization of the Newly Developed Scaffold

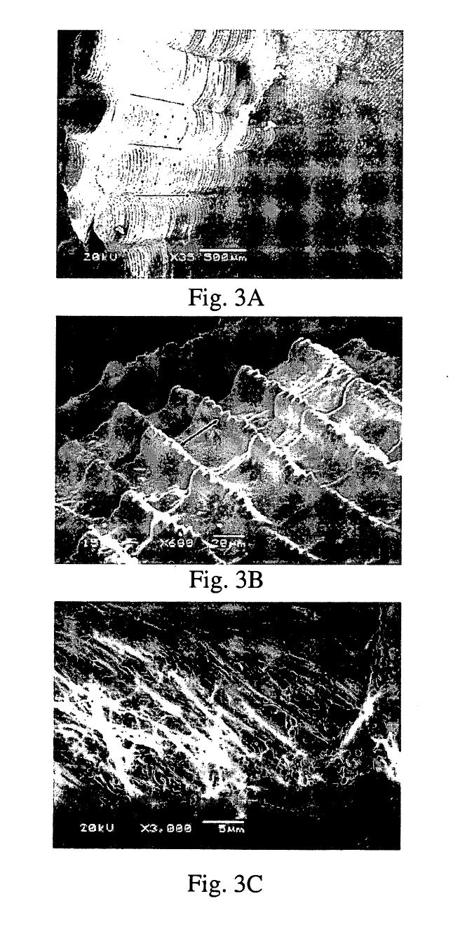

Scanning electron photomicrographs revealed a 3-dimensional (3-D), patterned scaffold with a microchannel-like structure (FIG. 3A). The widths of these microchannels had a range around 30 m (FIG. 3B), each individual channel possessing a uniform width along its entire length.

### Cytocompatibility Analysis on the Scaffold

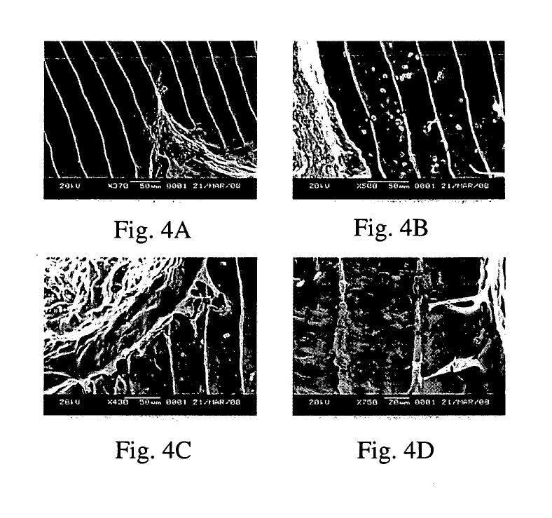

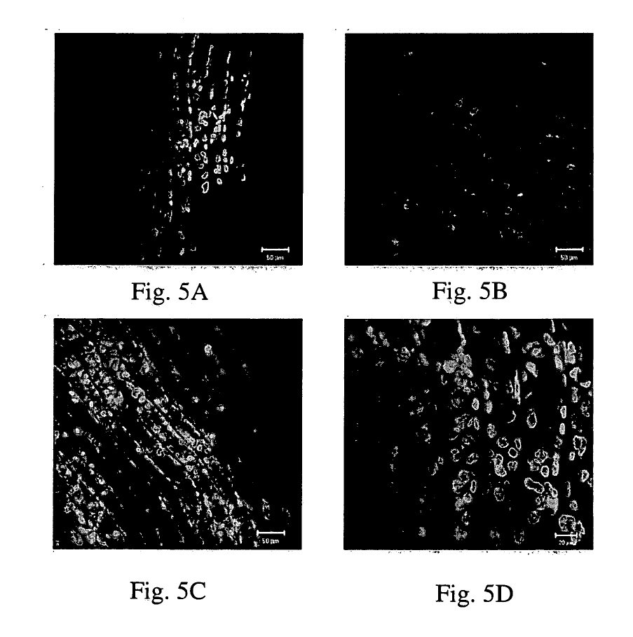

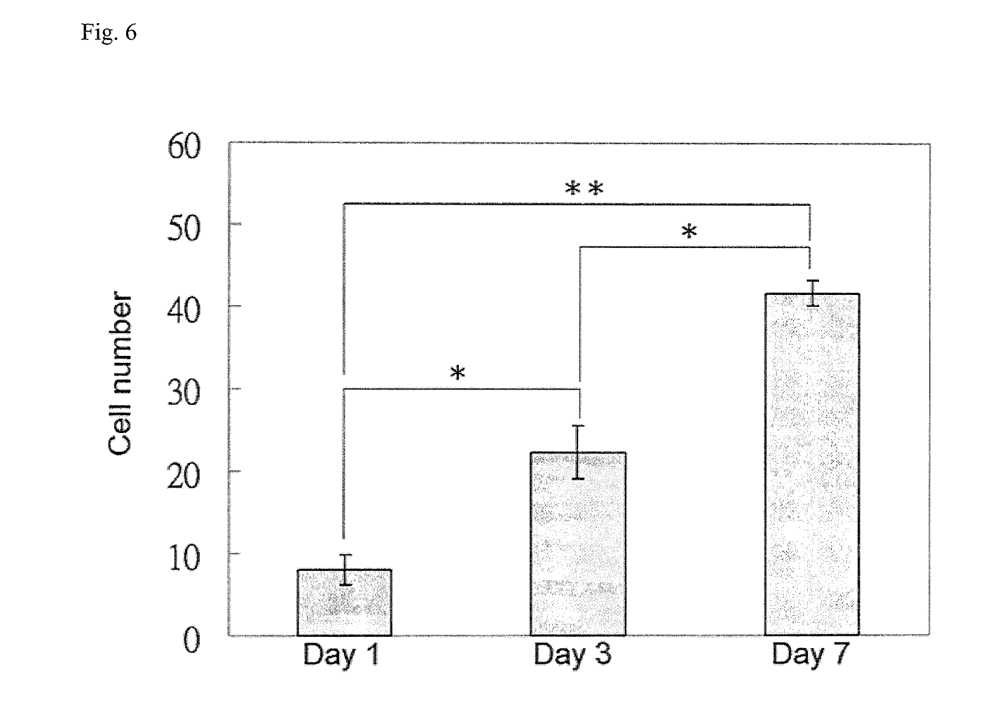

After seeding on the 3-D scaffolds with corneal cells, SEM demonstrated the orientation and morphology of corneal cells and their processes in parallel with the longitudinal guidance channels (FIG. 4). After seeding on the scaffolds, corneal cells rapidly attached to the scaffold surface (FIG. 4A). The cells extended long processes and migrated into the acellular decalcified scaffold (FIG. 4B). After 7 days of culture, corneal cells were shown to migrate deep into the scaffold and demonstrate a homogeneous and dense growth pattern with an orientation along the channels, indicating that the cells grow and migrate along the guidance channels structure of the scaffolds (FIG. 4D). In general, cell attachment, spreading and proliferation on the scaffold reflect the ability of the scaffold to make contact with the cells. Fewer proliferating cells on a substrate is a sign of weak cell-material interaction, which could be followed by cell death. To test the cytocompatibility of the scaffold with the corneal cells, cell attachment, spreading and morphology were observed under confocal microscopy. The cell morphology was visualized by confocal microscopy after 1 day, 2 days, 3 days and 7 days of culture. The micrograph (FIG. 5A) shows that cells on the scaffold surface after 1 day of culture were mostly round and fewer cells were attached onto the surface in comparison with the longer time periods of cultures. After 2 days of culture, the corneal cells distributed more densely, and contacted each other to form larger aggregates on the scaffold (FIG. 5B). Corneal cells proliferated exuberantly and started stacking in the guidance channels on the scaffold surfaces after more than 3 days of culture (FIGS. 5C and 5D). To assess the corneal cell growth on the scaffolds, The corneal cells were employed due to the fact that the cultured cells originated from the cell lines are easy to be dispersed and counted. The cells were seeded onto the scaffolds and cultured for various time periods. As determined by direct cell nuclei counting, the corneal cell proliferation on the scaffold was observed and showed a statistically significant increase in cell population during the 7-day culture period (FIG. 6).

Taken together, the results demonstrate that the fish scale-derived scaffold is cytocompatible with corneal cells and might be applied to production of tissue-engineering based artificial cornea. The results also suggest that the preserved micro-structure of the scaffold is advantageous to cell migration and spreading on the whole scaffold. The scaffold in corneal tissue engineering is attractive for the following reasons. First, acellular animal tissues are employed, which differ from hydrogels in being biodegradable. Second, the derived acellular material is hydrophilic highly cytocompatible with the host cells (FIGS. 4, 5) and with highly patterned structures (FIG. 3), that can readily promote cell conductive properties and bulk tissue integration for regenerating injured corneal tissues. Third, the derived acellular material is well permeable to gas, which is a prerequisite for materials using as cornea substitute.

In the invention, the method has developed to obtain an acellular, decalcified, fish scale-derived biodegradable scaffold with the long-term transparency, high mechanical strength, optimal biomechanical properties and regenerative capacity for artificial cornea development. The fabricated material keeps the naturally 3-D microstructure even after being acellularized and decalcified. The 3-D microstructure which is helpful for cell growth and migration since it guides cell populations to migrate in multiple parallel channels with spatial and functional reconstructions is also maintained. Moreover, the material is hydrophilic and permeable to oxygen. These properties make the scaffold to be a promising material for artificial cornea development. In summary, the present invention has demonstrated the feasibility of the fish scale-derived scaffold as a superior material for artificial cornea regeneration.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for preparing a biomaterial from a fish scale, comprising the steps of:
acellularizing the fish scale to remove a cell component, wherein the fish scale keeps the naturally 3-dimension microstructure after the step of acellularizing the fish scale;
cleaning the fish scale; and
decalcifying the fish scale, wherein in the step of acellularizing, it further including stirring constantly the fish scale in a solution with an octylphenoxypolyethoxyethanol; and rinsing in a Hanks' buffered saline.

2. The method according to claim 1, wherein in the step of cleaning the fish scale, it further includes running a Limulus Amebocyte Lysate (LAL) test; and repeating the step of cleaning the fish scale and running the LAL test until a value of LAL of the fish scale is below 200 EU/g. Even more preferred fish scale of the LAL test is less than 50 EU/g, and most preferably less than 20 EU/g.

3. The method according to claim 2, further comprising a step of dehydrating the fish scale after cleaning the fish scale and the fish scale is performed until the fish scale containing less than about 25% of water.

4. The method according to claim 1, further comprising a step of crosslinking the fish scale, so to chemically or physically reactive with an amine group or other reactive group in the biomaterials.

5. The method according to claim 1, further comprising a step of extruding the fish scale and the fish scale keeps the naturally 3-dimension microstructure after the step of extruding the fish scale.

6. The method according to claim 5, wherein the extrusion is performed at a temperature of less than about 200° C.

7. The method according to claim 5, further comprising a step of soaking the fish scale in water.

8. The method according to claim 1, wherein the step of acellularizing further comprises a step of soaking the fish scale in a hypotonic tris buffer that contains a protease inhibitor before the step of stirring in the solution.

9. The method according to claim 1, wherein the step of decalcifying is performed by immersing the fish scale in a solution comprised of an Ethylenediaminetetraacetic acid (EDTA) and a nitric acid.

10. A method for corneal regeneration, comprising steps of:
acellularizing the fish scale to remove a cell component, wherein the fish scale keeps the naturally 3-dimension microstructure after the step of acellularizing the fish scale;
cleaning the fish scale;
decalcifying the fish scale to serve as a scaffold;
harvesting a corneal;
seeding the corneal onto the scaffold in a plate; and
culturing a cell of the corneal, wherein in the step of acellularizing, it further includes stirring constantly the fish scale in a solution with an octylphenoxypolyethoxyethanol; and rinsing in a Hanks' buffered saline.

11. The method according to claim 11, wherein in the step of cleaning the fish scale, it further includes running a Limulus Amebocyte Lysate (LAL) test and repeating the step of cleaning the fish scale and running the LAL test until a value of LAL of the fish scale is below 200 Eu/mg.

12. The method according to claim 12, further comprising a step of dehydrating the fish scale after cleaning the fish scale and the fish scale is performed until the fish scale containing less than about 25% of water.

13. The method according to claim 11, further comprising a step of extruding the fish scale and the fish scale keeps the naturally 3-dimension microstructure after the step of extruding the fish scale.

14. The method according to claim 11, wherein the step of decalcifying is performed by immersing the fish scale in a solution comprised of an Ethylenediaminetetraacetic acid (EDTA) and a nitric acid.

15. The method according to claim 11, further comprising a step of rinsing the fish scale after decalcifying the fish scale.

16. The method according to claim 11, further comprising a step of dissecting the corneal into small pieces before seeding the corneal onto the scaffold in a plate.

17. The method according to claim 11, wherein the step of acellularizing further comprises a step of soaking in a hypotonic tris buffer that contains a protease inhibitor before the step of stirring in the solution.

18. The method according to claim 11, wherein the step of acellularizing further comprises a step of performing a digestion with a deoxyribonuclease (DNase) and a ribonuclease (RNase) after the step of stirring in the solution.

19. The method according to claim 11, wherein the step of culturing is performed by supplementing in a solution comprised of a Dulbecco's Modified Eagle's Medium, a fetal calf serum, a L-glutamine, a adenine, a antibiotic solution.
